(19) United States
(12) Patent Application Publication
(10) Pub. No.: US 2026/0090823 A1
(43) Pub. Date: Apr. 2, 2026

(54) METHOD AND DEVICE FOR AUTOMATICALLY DETERMINING THE POSITION OF A NEEDLE TIP OF A BIOPSY NEEDLE

(71) Applicant: Siemens Healthineers AG, Forchheim (DE)

(72) Inventors: Helmut GOLLWITZER, Erbendorf (DE); Sebastian NEWRZELLA, Bayreuth (DE); Ulf ZIMMERMANN, Frauenstein (DE); Mohd MAHMEEN, Bangalore (IN); Karin RATZMER, Hilpoltstein-Meckenhausen, (DE); Claus-Guenter SCHLIERMANN, Kemnath (DE); Konstantinos GKEKAS, Bayreuth (DE); Josef BOLTEN, Bonn (DE)

(73) Assignee: Siemens Healthineers AG, Forchheim (DE)

(21) Appl. No.: 19/341,314

(22) Filed: Sep. 26, 2025

(30) Foreign Application Priority Data
Sep. 27, 2024 (EP) 24203275.3

(51) Int. Cl. A61B 17/34; A61B 10/02; G06T 7/11

(52) U.S. Cl. CPC A61B 17/3403; A61B 10/0233; G06T 7/11; A61B 2562/08; G06T 2207/10004; G06T 2207/30068

(57) ABSTRACT

A method for automatically determining a position of a needle tip of a biopsy needle, which is fitted in a needle holder, configured to move on its longitudinal axis for a biopsy by a biopsy unit, and configured to be guided via a needle guide, comprises: positioning the biopsy needle in the needle holder; determining at least the position of the needle tip of the biopsy needle via a sensor; and automatically calculating a distance from the needle tip to a reference point.

### CROSS-REFERENCE TO RELATED APPLICATION(S)

The present application claims priority under 35 U.S.C. § 119 to European Patent Application No. 24203275.3, filed Sep. 27, 2024, the entire contents of which is incorporated herein by reference.

### FIELD

One or more example embodiments of the present invention relate to a method and a device for automatically determining the position of a needle tip of a biopsy needle as well as a mammography system.

### BACKGROUND

When a biopsy is used for diagnostic purposes, e.g., in mammography, core or vacuum needles are used, which range from around 90 mm to 150 mm in length. The needle parameters are often saved in a workstation that is being used, and the target position of a motorized movable biopsy unit is calculated and initiated using these needle parameters. For this purpose, the desired biopsy needle is inserted into a needle holder (also known as a “gun”), which can then be moved by a carriage of the biopsy unit, which is usually moved manually. The biopsy needle is then inserted into a patient by moving the needle holder on the carriage. Once the tip of the biopsy needle has reached the desired point, negative pressure is generated within it to draw a tissue sample into the biopsy needle (or also hereafter shortened to “needle”). The coordinates of the desired point are determined and calculated on the basis of multiple images typically via stereo calculation or reconstruction of a 3D dataset.

Once the needle holder has been pre-positioned, in practice the needle is typically inserted into the examination object, e.g., via a linear movement of the needle holder into the body. The needle is thereby guided by a sterile needle guide.

The position of the needle in the body is determined via repeated X-rays. However, this does subject the tissue to a radiation dose each time, which would be avoidable if the position of the needle was definitively known. Moreover, it must be ensured that the overall structure of the biopsy unit does not cast shadows on the images. Therefore, the needle is preferably arranged obliquely.

The needle is usually stopped before the desired point, e.g., a lesion, in order to check, e.g., via X-rays, whether the needle has been correctly positioned. If the needle body is inserted up to the end stop, then it is presumed that the desired precalculated position in the object (still before the lesion here) has been reached. The needle is then inserted to the end position for tissue removal via spring mechanisms, mechanical drives, or by vacuum and the tissue samples are taken.

Serious problems can arise if the needle is guided incorrectly or an incorrect needle type is used.

As needle manufacturers generally offer multiple needle lengths, allowing tissue samples to be removed at different depths in the examination object, if an incorrect needle is used there is a risk of missing the intended tissue or piercing through the object completely and thus causing injuries to the patient and damage to the examination device. If the system has been given, e.g., the wrong needle length, this can cause (potentially serious) injuries.

The needle length is only determined by manual measurement or by finding the length in the product data or customer documentation. The presence of a biopsy needle is currently detected through the presence of the needle holder or, e.g., a switch lever is flipped to insert the needle, wherein the lever is connected to a switch that blocks device and needle movements.

In summary, it can be stated that the main risk is positioning the needle tip incorrectly in the body. This incorrect positioning can result from the needle being inserted into the body incorrectly, or from the needle length not matching the information provided about needle length (incorrect needle inserted or incorrect information provided about needle length). Incorrect positioning can be detected through X-rays. However, these expose the patient's body to an X-ray dose and a correction has to be performed manually. Incorrect positioning can be overlooked entirely.

### SUMMARY

An object of one or more example embodiments of the present invention is to specify a method and a device for automatically determining the position of a needle tip of a biopsy needle as well as a mammography system, which will avoid the aforementioned disadvantages.

At least this object is realized by a method, a device, and a mammography system as claimed.

A method according to one or more example embodiments of the present invention is used to automatically determine the position of a needle tip of a biopsy needle, which is fitted in a needle holder and can be moved on its longitudinal axis for a biopsy by a biopsy unit and is guided via a needle guide. The method comprises the following steps:
positioning the biopsy needle in the needle holder,
determining at least the position of the needle tip of the biopsy needle via a sensor,
automatically calculating a distance from the needle tip to a reference point.

Information about this distance is then displayed as information to an operator and/or in the form of control commands. For example, a warning can be displayed that an incorrect needle has been inserted, or a movement of the needle holder can be blocked.

A biopsy needle is fitted in a needle holder for the method. This needle holder can be moved on its longitudinal axis (particularly on a sliding carriage) for a biopsy by a biopsy unit. The needle is guided by a (sterile) needle guide when moving. The needle is typically not yet in the needle guide straight after being inserted. It is only once the needle holder has been moved slightly that the needle passes through the needle guide and is then guided on its way by the needle guide. This is all well-known in the state of the art.

As regards the method steps, the distance determined there can be used both for determining the needle length and for determining the needle position. When determining both the length of the biopsy needle and the position of the biopsy needle, the position of the needle tip and the position of a reference point (e.g., on the needle holder or the needle guide) must be known. A position of the needle holder does not necessarily need to be measured, as it can also already be known in advance in its initial position due to the structure of the biopsy unit. When determining the position of the needle tip of the biopsy needle, X-rays can also be used throughout to monitor the process, but this is not strictly necessary.

The biopsy needle is first positioned in the known manner in the needle holder.

Next, at least the position of the needle tip of the biopsy needle is determined via a sensor. The position of the needle holder is preferably also determined. However, as stated, this is not strictly necessary because the position (e.g., in an operational position) can already be known.

Once the distance from the needle tip to the reference point has been determined, this can then be used both to determine the needle length (with a reference point on the needle holder) and to determine the needle position (with a fixed reference point in the room, e.g., on the needle guide). The distance from the needle tip to this reference point is thereby determined automatically. The reference point is preferably a point on the needle holder, e.g., its needle hold point. However, the reference point can also be the needle guide or another point outside the needle holder.

It is easy to calculate the total needle length with the information known in the method. It can be deduced from the distance between the needle tip and needle holder, e.g., from an image (a camera would be the sensor here). Admittedly, the needle length in the image is not that of the actual needle, but a conversion factor can easily be determined with a predefined acquiring distance and, where appropriate, a number of calibration measurements. Thus, only the pixels from the needle tip to the needle holder need actually be counted. The length of the actual needle is then calculated directly from the conversion factor.

As regards the position of the needle, the distance from the needle tip to a (known) reference point has to be calculated, e.g., on the needle guide or the needle holder. It should be noted here that it is not strictly necessary to measure the tip when determining the position of the needle tip of the biopsy needle. It is sufficient if the position can be deduced from measurements. It is preferable initially (if the needle tip is still outside the patient) for the position of the needle tip to be determined directly. This can preferably be achieved by a sensor on the needle guide determining its position on the needle guide. The position of the needle holder can then be measured, and the current position of the needle tip can be deduced (and determined as a result) from its position (with known needle length).

It is actually only necessary to run through the method steps once to measure the needle length. The method steps should be run through multiple times when determining the position of the needle tip in the body. It is particularly preferable for needle length to be determined before the biopsy needle is used and thus to verify whether the correct needle has been inserted. The position of the needle tip in the body can then be determined afterwards. The method steps after positioning the biopsy needle in the needle holder can also be substantiated as follows:
a) determining at least the position of the needle tip of a biopsy needle via a sensor, particularly in the area of the needle guide,
b) automatically calculating a total needle length from the needle tip to the needle holder as a first reference point,
c) determining the position of the needle holder via a sensor and determining the position of the needle tip of the biopsy needle from the position of the needle holder and the calculated total needle length,
d) automatically calculating a distance from the needle tip to the needle guide as a second reference point,
e) repeating steps c) and d) multiple times.

It should be noted that the potential movement vector of the needle holder in the biopsy unit is known. The needle holder can only be moved along this movement vector (or “movement direction”). As the biopsy needle is rigid, the needle tip is moved accordingly when the needle holder moves. Thus, if the position of a reference point on the needle holder is known and the distance from the needle to this reference tip is also known, then the position of the needle tip along the movement vector and therefore also in the room can be determined exactly.

A device according to one or more example embodiments of the present invention is used to automatically determine the position of a needle tip of a biopsy needle, which is fitted in a needle holder and can be moved on its longitudinal axis for a biopsy by a biopsy unit and is guided via a needle guide. The device comprises the following components:
optional: a sensor for detecting a position of a biopsy needle in the needle holder,
a determination unit designed to determine at least the position of the needle tip of the biopsy needle via a sensor,
a calculation unit designed to automatically calculate a distance from the needle tip to a reference point,
preferable: a data interface designed to display the distance, particularly in the form of a needle length or a position of the needle tip.

The function of the components of the device has already been described previously. The device is preferably designed to execute a method according to one or more example embodiments of the present invention.

A mammography system according to one or more example embodiments of the present invention comprises a device according to one or more example embodiments of the present invention and/or is designed to carry out a method according to one or more example embodiments of the present invention.

One or more example embodiments of the present invention can in particular be realized in the form of a computer unit with suitable software. The computer unit can have, e.g., one or more cooperating microprocessors or the like for this purpose. In particular, it can be realized in the form of suitable software program parts in the computer unit. Predominantly software-based realization has the advantage that even previously used computer units can be easily retrofitted to work in the manner, according to one or more example embodiments of the present invention, via a software or firmware update. In this respect, the object is also achieved through a corresponding computer program product with a computer program that can be loaded directly in a memory device of a computer unit, with program sections, in order to execute all steps of the method according to one or more example embodiments of the present invention, if the program is executed in the computer unit. In addition to the computer program, such a computer program product can comprise additional elements if applicable, e.g., documentation and/or additional components, including hardware components, e.g., hardware tools (dongles, etc.), for use of the software.

A computer-readable medium can be used, for example, a memory stick, a hard drive, or other transportable or integral data carrier, on which the program sections of the computer program are stored that can be read and executed by a computer unit, for transport to the computer unit or for storage on or in the computer unit.

Further particularly advantageous embodiments and developments of the present invention arise from the dependent claims and the description below, wherein the claims of one claim category can also be developed analogous to the claims and description sections of another claim category, and, in particular, individual features of different exemplary embodiments or variants can also be combined into new exemplary embodiments or variants.

A preferred sensor for determining the position of the needle tip of the biopsy needle within the scope of the method, or for a preferred device, is a capacitive sensor, an inductive sensor, an optical sensor, or an acoustic sensor. This sensor can directly measure the position of the needle tip or the position of the needle holder. However, this sensor preferably (directly) measures the position of the needle tip. This measurement is preferably taken via a sensor on or in the needle guide.

Alternatively or additionally, it is preferred that an optical sensor monitors whether the biopsy needle has reached a predefined position. It should be noted here that the needle tip always moves in the needle holder's direction of movement. An optical sensor, e.g., a light barrier can determine when the needle tip has reached a specific point in the direction of movement. Because the biopsy needle is generally metallic and, where appropriate, also magnetic, it is possible to determine the presence of the needle at a specific point using an inductive or capacitive sensor. A camera is also preferred as a sensor, as explained in more detail below.

The position of the needle holder is preferably measured at least if the needle tip is in a patient and/or after the needle length is determined or in addition to a (direct) measurement of the needle tip. A preferred sensor for this is a capacitive sensor, an inductive sensor, an optical sensor, or an acoustic sensor. A value can also be read from a potentiometer or its state can be measured with a sensor. It is also possible to determine the position of the needle holder from a control specification for moving the needle holder. The advantage of measuring the position of the needle holder in addition to (directly) measuring the position of the needle tip, is that the position of the needle tip can be determined even if the needle tip is in the patient.

The presence of the needle tip in the area of the needle guide is preferably verified first. If the needle tip was detected there, then it is no longer strictly necessary to continue determining the position of the needle tip. As the needle length is known (either determined automatically or because the inserted needle is known and verified) and the needle's movement direction is known (along the movement direction), the position of the needle tip can be determined unequivocally based on the determined position of the needle holder.

It is thereby preferable that a movement of the needle holder is read from a scale, or is determined via a scale, or that the state of a sliding carriage of the biopsy unit is determined. The state can be acquired via a control system or can also be visualized for the user on a display using computed acquisition, e.g., of the counting increments when using a scale or of the analog value when using analog measuring systems (e.g., potentiometers), starting when the needle is inserted into the needle detector, or by reading fixed positions. Furthermore, a programmable control system can evaluate position or needle length, adjust the correct position where appropriate, or trigger an error message. Adequate acquisition with lower accuracy can be achieved, e.g., by detecting a few positions, e.g., 3 preset lengths.

A measurement of the length of the biopsy needle is preferably taken before a biopsy and after positioning the biopsy needle in the needle holder. A measurement is preferably taken by acquiring an image of the biopsy needle in the needle holder (particularly via a camera) and evaluating the image. Alternatively, the position of the needle tip can also be determined on the needle guide, and the needle holder can then be moved in the direction of the needle guide until an anchor point (without patient). As already stated above, the advantage of measuring the length of the biopsy needle is that errors are avoided in determining the position of the needle tip, which arise from deviations between a specified needle length and an actual needle length.

The position of the needle tip of the biopsy needle is preferably determined via an optical sensor, preferably a camera (2D or 3D). It is thereby preferable that an image of the biopsy needle is acquired from a set acquiring position at a predefined distance from the biopsy needle and a length of the biopsy needle is calculated by finding the biopsy needle and the needle holder in the image and converting the length of the biopsy needle in the image (e.g., by counting pixels) to the true length of the biopsy needle via a predefined function (e.g., triangulation or a function to compensate for a known distortion in the image). The gauge of the biopsy needle in the image (e.g., also determined by counting pixels) is preferably also converted to the true gauge of the biopsy needle via a predefined function. This has the advantage that the length of the biopsy needle can be determined with very little effort and without having to move to a precise position. It should be noted here that the needles are only available in the form of (known) lengths, which are clearly distinct from each other. Therefore, it is generally not strictly necessary to determine the length with millimeter accuracy.

In one embodiment variant of the method, it is preferred that the presence of a biopsy needle in the needle holder is determined via a sensor. Such a sensor can be, e.g., a switch, which is activated if there is a biopsy needle in the needle holder and deactivated if not. A needle holder is then blocked from moving in predefined situations when a biopsy needle has been inserted, particularly if a distance from the needle tip to a fixed reference point exceeds a predefined limit. This prevents the biopsy needle from penetrating beyond the target area in a patient.

The following steps are preferably carried out after positioning the biopsy needle in the needle holder and preferably also after optionally verifying whether a biopsy needle has been inserted into the needle holder:
taking an image of the biopsy needle and the needle holder,
segmenting the image,
carrying out contour detection at least of the biopsy needle,
counting those pixels that have been segmented as the biopsy needle,
calculating the length of the actual biopsy needle via the length of the biopsy needle in the image and a predefined formula and preferably also the gauge of the actual biopsy needle via the gauge of the biopsy needle in the image and a predefined formula,
displaying the calculated values, preferably in the form of an image with corresponding dimensions.

The image is preferably acquired by a camera, as has already been described above. It is thereby important that the image includes the entire biopsy needle and at least the part of the needle holder in which the biopsy needle is mounted.

Segmenting an image is known in the state of the art. This is preferably effected via a model capable of machine-learning that has been trained to detect biopsy needles and needle holders in an image. After this segmentation, it is known which pixels in the image represent the biopsy needle and which represent the needle holder.

Carrying out contour detection of segmented elements (i.e., also of the biopsy needle) is known in the state of the art. This is preferably effected via a minimal bounding rectangle method, which is likewise known in the state of the art. This finds the smallest rectangle around the contour in question. The pixels representing the biopsy needle are thus separated

These separated pixels can then be counted. It is simplest if the acquisition is aligned so that the needle runs precisely in the X or Y-direction in the image. Oblique views are also entirely possible. The count is preferably effected at least in the vertical direction. The count is used to determine the length of the biopsy needle in the image. Pixels are preferably also counted in the horizontal direction. This count is used to determine the gauge of the biopsy needle in the image.

Once the pixel count is known, the length of the actual biopsy needle is calculated. As the biopsy needle in the image has a different length to that in reality, but this is merely scaled by a known (or determinable) function, the actual length can be calculated via the length of the biopsy needle in the image and a predefined formula. In the event that there is a linear relationship, the actual length would be L=aB with the factor a and the length B in the image. The gauge of the actual biopsy needle is also preferably calculated via the gauge of the biopsy needle in the image and a predefined formula. This can be done in the same way as the length. However, it should be noted here that the biopsy needle is often arranged obliquely. This affects both the length and the gauge calculation (the needle would be slightly conical here). However, this only has a minor impact in practice, as the inclination can be compensated in the length calculation by choosing the appropriate function. As regards the gauge, a specific point of the needle can be selected (e.g., the tip).

The calculated values can then, e.g., be displayed or stored in a log file. It is preferable if the image of the needle is displayed with corresponding dimensions.

According to one preferred embodiment variant of the method, the needle holder is also examined for features disclosing its manufacturer. The image is thereby preferably examined for signs at the position of the needle holder, particularly in the form of logos and/or characters. Found characters are then compared to a list of signs. This improves the verification of whether a correct needle has been used.

A preferred device comprises an optical sensor designed and arranged to acquire images of the needle holder and an inserted biopsy needle. The device also comprises a length determination unit designed to determine the length of the biopsy needle. It is particularly preferably designed to find the biopsy needle and the needle holder in the image and to convert the length of the biopsy needle in the image to the true length of the biopsy needle via a predefined function. It is preferably also designed to convert the gauge of the biopsy needle in the image to the true gauge of the biopsy needle via a predefined function.

According to one preferred embodiment variant of the method, before the biopsy needle is inserted into the needle holder, an identification code, e.g., a QR code, a label, a character string, NFC information, or a barcode, is scanned on the biopsy needle or on packaging of the biopsy needle. The scanned information is then compared to predefined information about a desired needle type. If the identification code indicates that this is not the desired needle type, a warning is displayed. This has the advantage of preventing the packaging of a sterile needle being ripped open erroneously. Alternatively or additionally, the biopsy needle is blocked from being inserted into the needle holder. This has the advantage that, in addition to human attentiveness, there is another safeguard against the use of an incorrect needle.

A preferred device comprises a sensor designed to detect the needle tip on the needle guide and a sensor designed to determine the position of the needle holder. It is thereby preferred that the calculation unit is designed, based on the measured position of the needle tip on the needle guide and the position of the needle holder, to automatically calculate a distance from the needle tip to a fixed reference point in the room, preferably to a point on the needle holder.

It may be desired to only determine needle length to verify whether the correct biopsy needle is present. A preferred method for this purpose is used to automatically determine the length of a biopsy needle. The method comprises the following steps:
providing a biopsy needle (in its packaging or in the needle holder),
acquiring an image of the biopsy needle (in the needle holder or in its packaging) with an optical sensor, particularly a camera or a code reader, or acquiring NFC information for the needle via a sensor,
determining the needle length from the sensor information, particularly the image information from the image,
displaying the needle length (as information for an operator or in the form of control commands).

Two embodiment variants of the method are thereby particularly preferred. For one thing, this method allows for a code (e.g., barcode or QR code) on the biopsy needle or packaging to be read and verified to determine whether a correct (packaged) biopsy needle has been selected. This embodiment variant can prevent an incorrect biopsy needle being inserted erroneously, which would then need to be disposed of as it would no longer be sterile. Thus, this embodiment variant prevents unnecessary costs.

The other preferred embodiment variant allows the needle length of an inserted needle to be determined automatically from the images. This has already been described above.

First, the biopsy needle is provided. It can be present in its packaging (for the one preferred method) or already be in the needle holder (for the other preferred method).

An image of the biopsy needle is then acquired. For the one preferred method, this can be the image of a reader for barcodes or QR codes and, for the other preferred method, an image of a camera as described above.

The needle length can then be determined from this image. With the one preferred method, the needle length can be read directly from the information in the code and, with the other preferred method, by evaluating the image information (e.g., counting pixels) as already described above.

The determined needle length can then be displayed as information to an operator in the form of control commands.

A preferred device in this regard for automatically determining the length of a biopsy needle, particularly with the method described above, comprises the following components:
providing a biopsy needle (in its packaging or in the needle holder),
an optical sensor designed to acquire an image of the biopsy needle, particularly a camera or a code reader,
a length determination unit designed to determine the needle length from image information from the image,
a data interface designed to display the needle length (as information for an operator or in the form of control commands).

The function of the components of the device has already been described previously. The device is preferably designed to execute a method according to one or more example embodiments of the present invention.

A preferred mammography system comprises this device and/or is designed to carry out the preferred method.

A preferred sensor for the preferred method or the preferred device for measuring length was already described above. The same applies to monitoring whether a biopsy needle has reached a predefined position.

A preferred measurement of needle length is preferably taken by acquiring an image of the biopsy needle in the needle holder (particularly via a camera) and evaluating the image. Alternatively, the position of the needle tip can also be determined on the needle guide, and the needle holder can then be moved in the direction of the needle guide until an anchor point (without patient). As already stated above, the advantage of measuring the length of the biopsy needle is that errors are avoided in determining the position of the needle tip, which arise from deviations between a specified needle length and an actual needle length.

As stated, it is preferred for the needle length to be determined via an optical sensor, preferably a camera (2D or 3D). It is thereby preferable that an image of the biopsy needle is acquired from a set acquiring position at a predefined distance from the biopsy needle and a length of the biopsy needle is calculated by finding the biopsy needle and the needle holder in the image and converting the length of the biopsy needle in the image (e.g., by counting pixels) to the true length of the biopsy needle via a predefined function (e.g., triangulation or a function to compensate for a known distortion in the image). The gauge of the biopsy needle in the image (e.g., also determined by counting pixels) is preferably also converted to the true gauge of the biopsy needle via a predefined function. This has the advantage that the length of the biopsy needle can be determined with very little effort and without having to move to a precise position. It should be noted here that the needles are only available in the form of (known) lengths, which are clearly distinct from each other. Therefore, it is generally not strictly necessary to determine the length with millimeter accuracy.

The following steps are preferably carried out after positioning the biopsy needle in the needle holder and preferably also after optionally verifying whether a biopsy needle has been inserted into the needle holder:
acquiring an image of the biopsy needle and the needle holder,
segmenting the image,
carrying out contour detection at least of the biopsy needle,
counting those pixels that have been segmented as the biopsy needle,
calculating the length of the actual biopsy needle via the length of the biopsy needle in the image and a predefined formula and preferably also the gauge of the actual biopsy needle via the gauge of the biopsy needle in the image and a predefined formula,
displaying the calculated values, preferably in the form of an image with corresponding dimensions.

The image is preferably acquired by a camera, as has already been described above. It is thereby important that the image includes the entire biopsy needle and at least the part of the needle holder in which the biopsy needle is mounted.

Segmenting an image is known in the state of the art. This is preferably effected via a model capable of machine-learning that has been trained to detect biopsy needles and needle holders in an image. After this segmentation, it is known which pixels in the image represent the biopsy needle and which represent the needle holder.

Carrying out contour detection of segmented elements (i.e., also of the biopsy needle) is known in the state of the art. This is preferably effected via a minimal bounding rectangle method, which is likewise known in the state of the art. This finds the smallest rectangle around the contour in question. The pixels representing the biopsy needle are thus separated

These separated pixels can then be counted. It is simplest if the acquisition is aligned so that the needle runs precisely in the X or Y-direction in the image. Oblique views are also entirely possible. The count is preferably effected at least in the vertical direction. The count is used to determine the length of the biopsy needle in the image. Pixels are preferably also counted in the horizontal direction. This count is used to determine the gauge of the biopsy needle in the image.

Once the pixel count is known, the length of the actual biopsy needle is calculated. As the biopsy needle in the image has a different length to that in reality, but this is merely scaled by a known (or determinable) function, the actual length can be calculated via the length of the biopsy needle in the image and a predefined formula. In the event that there is a linear relationship, the actual length would be L=aB with the factor a and the length B in the image. The gauge of the actual biopsy needle is also preferably calculated via the gauge of the biopsy needle in the image and a predefined formula. This can be done in the same way as the length. However, it should be noted here that the biopsy needle is often arranged obliquely. This affects both the length and the gauge calculation (the needle would be slightly conical here). However, this only has a minor impact in practice, as the inclination can be compensated in the length calculation by choosing the appropriate function. As regards the gauge, a specific point of the needle can be selected (e.g., the tip).

The calculated values can then, e.g., be displayed or stored in a log file. It is preferable if the image of the needle is displayed with corresponding dimensions.

According to one preferred embodiment variant of the method, the needle holder is also examined for features disclosing its manufacturer. The image is thereby preferably examined for signs at the position of the needle holder, particularly in the form of logos and/or characters. Found characters are then compared to a list of signs. This improves the verification of whether a correct needle has been used.

A preferred device comprises, as stated, an optical sensor designed and arranged to acquire images of the needle holder and an inserted biopsy needle. The device also comprises a length determination unit designed to determine the length of the biopsy needle. It is particularly preferably designed to find the biopsy needle and the needle holder in the image and to convert the length of the biopsy needle in the image to the true length of the biopsy needle via a predefined function. It is preferably also designed to convert the gauge of the biopsy needle in the image to the true gauge of the biopsy needle via a predefined function.

According to one preferred embodiment variant of the method, before the biopsy needle is inserted into the needle holder, an identification code, e.g., a QR code, a label, a character string, NFC information, or a barcode, is scanned on the biopsy needle or on packaging of the biopsy needle. The scanned information is then compared to predefined information about a desired needle type. If the identification code indicates that this is not the desired needle type, a warning is displayed. This has the advantage of preventing the packaging of a sterile needle being ripped open erroneously. Alternatively or additionally, the biopsy needle is blocked from being inserted into the needle holder. This has the advantage that, in addition to human attentiveness, there is another safeguard against the use of an incorrect needle.

The use of AI-based methods is preferred (AI: artificial intelligence) for the method according to one or more example embodiments of the present invention. Artificial intelligence is based on the principle of machine-based learning and is generally carried out with an adaptive algorithm that has been trained appropriately. Machine-based learning is often referred to simply as machine learning, which also includes the principle of deep learning. Finding the biopsy needle in images is particularly a task for such a system.

Components of one or more example embodiments of the present invention are preferably available as a cloud service. Such a cloud service is used to process data, particularly via an artificial intelligence, but can also be a service based on conventional algorithms or a service in which an evaluation is performed in the background by people. A cloud service (hereafter also shortened to “cloud”) is generally an IT infrastructure, providing, e.g., memory or computing power and/or application software, via a network. Communication between the user and the cloud is effected via data interfaces and/or data transmission protocols. In the present case, it is particularly preferred for the cloud service to provide both computing power and application software.

Data that was obtained within the scope of one or more example embodiments of the present invention is provided via a network to the cloud service within the scope of a preferred method. This comprises a computing system, which generally does not comprise the user's local computer. The method can thereby be realized via a command configuration in a network. The data calculated in the cloud is later sent back over the network to the user's local computer.

### BRIEF DESCRIPTION OF THE FIGURES

The present invention is explained again in more detail below with reference to the attached figures based on exemplary embodiments. The different figures feature the same components with identical reference numbers. The figures are not generally to scale. They show:

FIG. 1 a rough schematic representation of a preferred mammography system with a preferred device,

FIG. 2 a biopsy unit

FIG. 3 a preferred sensor arrangement on a needle holder equipped with a biopsy needle,

FIG. 4 another preferred sensor arrangement on a needle holder equipped with a biopsy needle,

FIG. 5 the needle length being measured with a camera,

FIG. 6 the sequence of the method as a block diagram,

FIG. 7 a barcode being scanned to determine the needle type.

### DETAILED DESCRIPTION

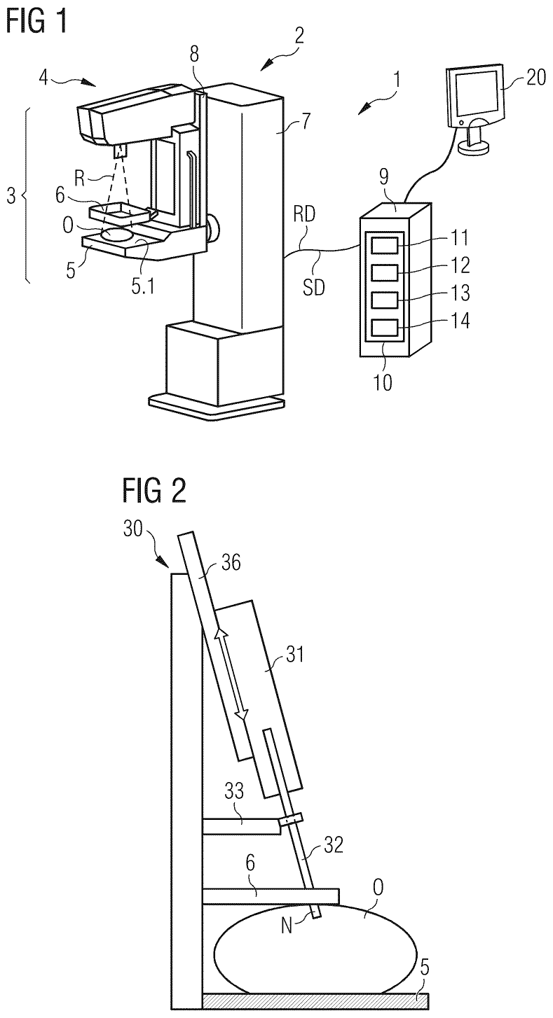

FIG. 1 shows a rough schematic of an exemplary mammography system 1 in the form of a tomosynthesis system 1. Relative direction information, such as “top,” “bottom,” etc., relates to a tomosynthesis system 1 intentionally positioned for operation. The tomosynthesis system 1 comprises a tomosynthesis machine 2 and a control device 9.

The tomosynthesis machine 2 has a stand 7 and a source-detector arrangement 3, which comprise in turn an X-ray tube assembly 4 and a detector 5 with a detector surface 5.1. The stand 7 stands on the ground in operation. It is movably connected to the source-detector arrangement 3 so that the height of the detector surface 5.1, i.e., the distance to the ground, can be adjusted to a chest height of a patient.

A breast O of the patient (shown schematically here) rests on the upper side of the detector surface 5.1 as the examination object O for an examination. A compression plate 6 is arranged above the breast O and the detector surface 5.1, which is movably connected to the source-detector arrangement 3. The breast O is simultaneously compressed and fixed in place for the examination by the compression plate 6 being lowered onto it so that a pressure is exerted on the breast O between the compression plate 6 and the detector surface 5.1. The contact surface of the compression plate 6 facing the breast O has a concave curvature so that the breast curves convexly there, as shown in FIGS. 4, 5, and 6, for example.

The X-ray tube assembly 4 is arranged and designed in such a way with respect to the detector 5 that the detector 5 senses X-rays R emitted from it, once at least some of the X-rays R have penetrated the breast O of the patient. The X-ray tube assembly 4 can thereby be swiveled relative to the detector 5 via a rotating arm 8 in a range of ±50° about a basic position, in which it stands perpendicular above the detector surface 5.1. The section to be scanned can be predefined or limited via a collimator C.

The control device 9 obtains the raw data RD of the measurement and sends control data SD to the tomosynthesis machine 2 via a data interface. It is connected to a terminal 20, through which a user can give the tomosynthesis system 1 commands or retrieve measurement results. The control device 9 can be arranged in the same room as the tomosynthesis machine 2 but can also be located in an adjacent control room or at an even greater spatial distance.

The device 10, according to one or more example embodiments of the present invention, is used to automatically determine the position of a needle tip N of a biopsy needle 32, which is fitted in a needle holder 31 and can be moved on its longitudinal axis for a biopsy by a biopsy unit 30 and is guided via a needle guide 33 (see FIG. 2.). It comprises a determination unit 11, a calculation unit 12, and a length determination unit 13. A sensor for detecting a position of a biopsy needle 32 in the needle holder 31 is also preferred (not shown). The device is arranged in the control device here. However, it can also be useful in other positions, e.g., on the rotating arm 8 or at another point near the X-ray R or even directly on the biopsy unit 30.

The determination unit 11 is used to determine at least the position of the needle tip N of the biopsy needle 32 via a sensor 35 (see following figures).

The calculation unit 12 is used to automatically calculate a distance from the needle tip N to a reference point.

The length determination unit 13 is used to determine the length of the biopsy needle 32, preferably by finding the biopsy needle 32 and the needle holder 31 in the image B and converting the length of the biopsy needle 32 in the image B to the true length of the biopsy needle 32 via a predefined function. It can also be used to convert the gauge of the biopsy needle 32 in the image B to the true gauge of the biopsy needle 32 via a predefined function.

The result image E is then displayed via a data interface 14.

FIG. 2 shows a biopsy unit 30 with a needle holder 31, which is arranged on a sliding carriage 36. A biopsy needle 32 is inserted into the needle holder 31, which is guided by a needle guide 33. The biopsy needle 32 has been inserted here into an examination object O, here a female breast O, to remove a biopsy. The breast O thereby rests on a detector 5 of a mammography system 1 according to FIG. 1, and the biopsy needle 32 has been passed through a hole in the compression plate 6. A double arrow indicates the possible direction of movement of the biopsy needle 32 and needle holder 31.

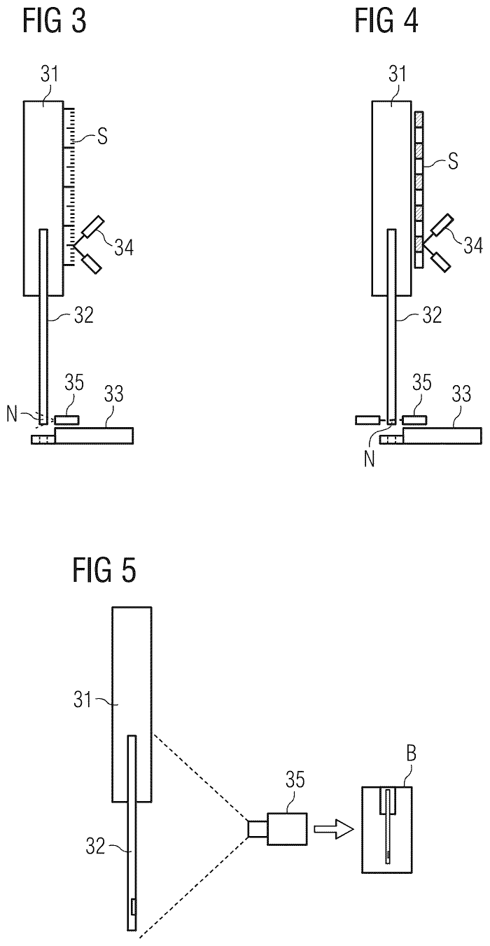

FIGS. 3 and 4 show a preferred sensor arrangement on a needle holder 31 equipped with a biopsy needle 32 of a biopsy unit 30 according to FIG. 2. Both embodiment variants show in each case a sensor 34, which monitors the movement of the needle holder, and a sensor 35 on the needle guide 33, which is intended to detect the position of the needle tip N.

In FIG. 3, a scale S is applied to the needle holder, whose position is monitored by an optical sensor 34. The position of the needle holder 31 can thus be determined very accurately; as the configuration of needle holder 31 and biopsy needle 32 is rigid, the position of the biopsy needle 32 can be inferred directly from the position of the needle holder 31. The needle tip N on the needle holder 31 is also determined to be safe, however. This ensures that a specific position on the scale truly matches a (determined) position of the needle tip N. If, for example, an incorrect needle length has been selected, this would then be detected. The needle holder 31 can also be moved to a predefined, rear position (where the needle tip N cannot yet be detected on the needle holder 31 and can then be advanced slowly, until the sensor 35 on the needle holder 31 detects the needle tip N. The position of the scale S can now be correlated directly to the position of the needle tip N, and even the needle length can be determined. The sensor shown here can be an inductive, capacitive, or acoustic sensor, for example.

In FIG. 4, a shaded scale S is applied to the needle holder, whose position is monitored by an optical sensor 34. The position of the needle holder 31 can thus also be determined very accurately, particularly if the sensor 34 has a spatial resolution. The needle tip N on the needle holder 31 is also determined to be safe here: in this example with an optical sensor 35 in the form of a light barrier.

FIG. 5 shows the position of the needle tip of the biopsy needle 32 being determined via an optical sensor 35 in the form of a camera. In this example, an image B of the biopsy needle 32 is acquired from a set acquiring position at a predefined distance from the biopsy needle 32. A length of the biopsy needle 32 is then calculated from this image, by finding the biopsy needle 32 and the needle holder 31 in the image B and converting the length of the biopsy needle 32 in the image B to the true length of the biopsy needle 32 via a predefined function.

This is preferably effected via the steps:
acquiring an image B of the biopsy needle 32 and the needle holder 31,
segmenting the image B, preferably via a model capable of machine-learning trained to detect biopsy needles 32 and needle holders 31 in an image B,
carrying out contour detection at least of the biopsy needle 32, preferably via a minimal bounding rectangle method
counting those pixels that have been segmented as the biopsy needle 32, preferably at least in the vertical direction to determine the length of the biopsy needle 32 in the image B, preferably also in the horizontal direction to determine the gauge of the biopsy needle 32 in the image B,
calculating the length of the actual biopsy needle 32 via the length of the biopsy needle 32 in the image B and a predefined formula and preferably also the gauge of the actual biopsy needle 32 via the gauge of the biopsy needle 32 in the image B and a predefined formula,
displaying the calculated values, preferably in the form of an image B with corresponding dimensions.

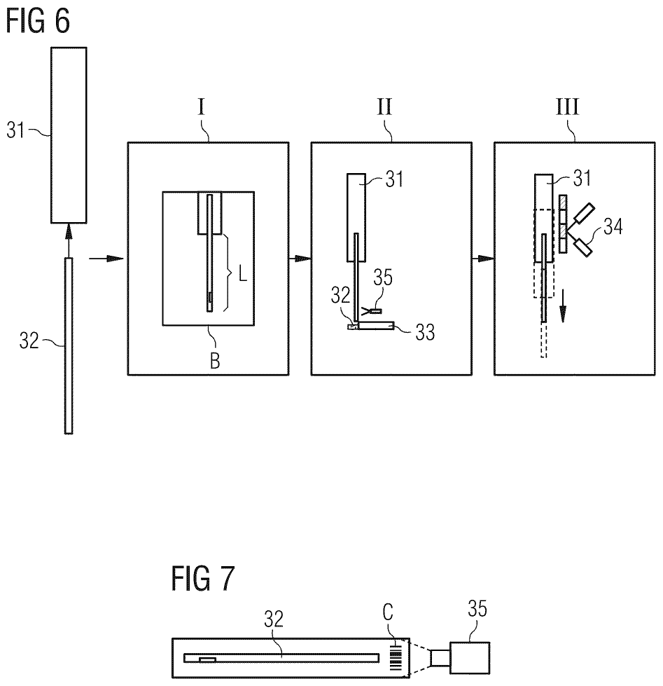

FIG. 6 shows the sequence of the method for automatically determining the position of a needle tip of a biopsy needle with a biopsy unit according to FIG. 2 as a block diagram.

First, a biopsy needle 32 is positioned in the needle holder 31 (left).

In step I, the length L of the biopsy needle 32 is then determined initially from an image as described for FIG. 5.

In step II, at least the position of the needle tip N of the biopsy needle 32 on the needle guide 33 is determined via a sensor 35.

In step III, a distance from the needle tip N to the needle guide 33 is calculated automatically as a reference point. The position of the needle holder 31 is thereby determined by a sensor 34 and its exact position is inferred from the determined length L of the biopsy needle 32.

FIG. 7 shows a barcode being scanned to determine the needle type. Before the biopsy needle 32 is inserted into the needle holder 31, a barcode on the biopsy needle 32 or packaging of the biopsy needle 32 is thereby scanned. The scanned information is then compared to predefined information about a desired needle type. If the barcode C indicates that this is not the desired needle type, a warning is displayed.

Finally, it is noted once again that the present invention described in detail above merely concerns exemplary embodiments, which can be modified by the person skilled in the art in a wide variety of ways without leaving the field of the present invention. Moreover, use of the indefinite article “a” or “an” does not prevent the features concerned being present multiple times. Likewise, terms like “unit” do not exclude the possibility that the components concerned comprise multiple interacting subcomponents, which may be distributed, including spatially if applicable. The term “a number” should be read as “at least one”. Gender-neutral language is used throughout this text.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A method for automatically determining a position of a needle tip of a biopsy needle, which is fitted in a needle holder, configured to move on a longitudinal axis for a biopsy by a biopsy unit, and configured to be guided via a needle guide, the method comprising:
positioning the biopsy needle in the needle holder;
determining, via a sensor, at least the position of the needle tip of the biopsy needle; and
automatically calculating a distance from the needle tip to a reference point.

2. The method as claimed in claim 1, wherein the sensor is a capacitive sensor, an inductive sensor, an optical sensor, or an acoustic sensor.

3. The method as claimed in claim 1, wherein at least one of when the needle tip is in a patient or after a needle length is determined, the method further comprises:
measuring a position of the needle holder via a capacitive sensor, an inductive sensor, an optical sensor, or an acoustic sensor, or from a state of a potentiometer or a control specification.

4. The method as claimed in claim 1, wherein, before the biopsy and after positioning the biopsy needle in the needle holder, the method further comprises:
measuring a length of the biopsy needle by acquiring an image of the biopsy needle in the needle holder and evaluating the image, or by determining a position of the needle tip on the needle guide and moving the needle holder in a direction of the needle guide until an anchor point is reached.

5. The method as claimed in claim 4, wherein the position of the needle tip of the biopsy needle is determined via an optical sensor.

6. The method as claimed in claim 1, wherein presence of the biopsy needle in the needle holder is determined via a another sensor, and the needle holder is blocked from moving in defined situations when a biopsy needle is inserted.

7. The method as claimed in claim 5, wherein after positioning the biopsy needle in the needle holder and verifying whether a biopsy needle is inserted into the needle holder, the method further comprises:
acquiring an image of the biopsy needle and the needle holder;
segmenting the image via a model capable of machine-learning trained to detect biopsy needles and needle holders in images;
carrying out contour detection at least of the biopsy needle;
counting pixels that have been segmented as the biopsy needle at least in a vertical direction to determine the length of the biopsy needle in the image;
calculating an actual length of the biopsy needle via the length of the biopsy needle in the image and a first formula; and
displaying the actual length in the form of an image with corresponding dimensions.

8. The method as claimed in claim 5, wherein the needle holder is examined for features identifying a manufacturer.

9. The method as claimed in claim 1, wherein, before the biopsy needle is inserted into the needle holder, the method further comprises:
scanning an identification code on the biopsy needle or packaging of the biopsy needle;
comparing information obtained from the identification code with information about a desired needle type; and
in response to the identification code indicating that the biopsy needle is not the desired needle type, at least one of displaying a warning or blocking the biopsy needle from being inserted into the needle holder.

10. A device for automatically determining a position of a needle tip of a biopsy needle, which is fitted in a needle holder, movable on a longitudinal axis for a biopsy by a biopsy unit, and guided via a needle guide, wherein the device comprises:
a determination unit configured to determine at least a position of the needle tip of the biopsy needle via a sensor; and
a calculation unit configured to automatically calculate a distance from the needle tip to a reference point.

11. The device as claimed in claim 10, further comprising:
an optical sensor configured to acquire images of the needle holder and the biopsy needle;
a length determination unit configured to determine a length of the biopsy needle based on the images.

12. The device as claimed in claim 10, further comprising:
a sensor configured to detect the needle tip on the needle guide; and
a sensor configured to determine a position of the needle holder, wherein
the calculation unit is configured to, based on the position of the needle tip on the needle guide and the position of the needle holder, automatically calculate a distance from the needle tip to a fixed reference point in a room, and
the fixed reference point is a point on the needle holder.

13. A mammography system comprising:
the device as claimed in claim 10.

14. A non-transitory computer program product comprising commands that, when executed by a computer, cause the computer to carry out the method as claimed in claim 1.

15. A non-transitory computer-readable storage medium comprising commands that, when executed by a computer, cause the computer to carry out the method as claimed in claim 1.

16. The method as claimed in claim 1, wherein at least one of the sensor is on or in the needle guide or the sensor is an optical sensor configured to monitor whether the biopsy needle has reached a first position.

17. The method as claimed in claim 3, further comprising:
reading a movement of the needle holder from a scale,
determining the movement of the needle holder via the scale, or
determining a state of a sliding carriage of the biopsy unit.

18. The method as claimed in claim 5, wherein
the optical sensor is a camera, and
an image of the biopsy needle is acquired from a set acquiring position at a first distance from the biopsy needle,
a length of the biopsy needle is calculated by finding the biopsy needle and the needle holder in the image and converting the length of the biopsy needle in the image to a true length of the biopsy needle via a first function, and
a gauge of the biopsy needle in the image is converted to a true gauge of the biopsy needle via a second function.

19. The method as claimed in claim 6, wherein the defined situations include a distance from the needle tip to a fixed reference point exceeding a threshold limit.

20. The method as claimed in claim 7, wherein after positioning the biopsy needle in the needle holder and verifying whether the biopsy needle is inserted into the needle holder, the method further comprises:
counting pixels that have been segmented as the biopsy needle in a horizontal direction to determine a gauge of the biopsy needle in the image;
calculating an actual gauge of the biopsy needle via the gauge of the biopsy needle in the image and a second formula,
displaying the actual gauge in the form of an image with corresponding dimensions.

21. The method as claimed in claim 8, wherein the needle holder is examined for features identifying a manufacturer by
examining the image for signs at the position of the needle holder in the form of at least one of logos or characters, and
comparing found characters to a list of signs.

22. The method as claimed in claim 9, wherein the identification code includes at least one of a QR code, a label, a character string, NFC information or a barcode.

23. The device as claimed in claim 10, further comprising:
the sensor configured to detect a position of the biopsy needle in the needle holder.

24. The device as claimed in claim 11, wherein the length determination unit is configured to determine the length of the biopsy needle by finding the biopsy needle and the needle holder in the images and converting the length of the biopsy needle in the images to a true length of the biopsy needle via a first function.

25. The device as claimed in claim 24, wherein the length determination unit is configured to convert a gauge of the biopsy needle in the images to a true gauge of the biopsy needle via a second function.
